(19) United States
(12) Patent Application Publication
(10) Pub. No.: US 2025/0281661 A1
(43) Pub. Date: Sep. 11, 2025

(54) EMULSIONS FOR USE IN AIR FRESHENERS DEVICES

(71) Applicant: Symrise AG, Holzminden (DE)

(72) Inventors: Astrid KAUFHOLD, Holzminden (DE); Julia VON HOLLY-PONIENTZIETZ, Holzminden (DE); Falk HARZKE, Rühle (DE); Christine WINGERTER, Holzminden (DE)

(21) Appl. No.: 18/861,258

(22) PCT Filed: May 4, 2022
(86) PCT No.: PCT/EP2022/061978

§ 371 Date: Oct. 29, 2024

(51) Int. Cl. A61L 9/12; A61L 9/04; C11B 9/00

(52) U.S. Cl. CPC A61L 9/127; A61L 9/044; C11B 9/0003

(57) ABSTRACT

The present invention relates to liquid air fresheners devices and to specific emulsions for use in said liquid air fresheners devices.

The present invention relates to liquid air fresheners devices and to specific emulsions for use in said liquid air fresheners devices.

### BACKGROUND

Air fresheners are consumer products that typically emit fragrance and are used in homes or commercial interiors such as restrooms, foyers, hallways, vestibules and other smaller indoor areas, as well as larger areas such as hotel lobbies, auto dealerships, spa facilities, public arenas and other large interior spaces.

Many liquid air fresheners are commercially available in different formats such as reed or wick diffusers, electrical plug-in devices, aerosols, or sprays. The perfume composition of the air freshener may be a true solution, or a colloidal solution such as a microemulsion.

EP 2629807 B1 claims a liquid reed diffusor fragrance composition comprising: (a) 0.1% to 60% by weight of an oil phase comprising a fragrance; (b) at least 10% by weight of water; (c) a fragrance vehicle comprising at least one mono-, di-, or polyglycol ether; and (d) an amphiphilic solubilizing agent.

EP 2810663 B1 describes a fragrance composition comprising at least two different fragrance accords wherein at least 30 wt.-percent of each fragrance accord comprises the key contributor(s) of said fragrance accord and wherein the average of the odor detection thresholds of said key contributor(s) for each fragrance accord is within the same order of magnitude as the average of the odor detection thresholds of the key contributor(s) for said other fragrance accord(s); and wherein the base note(s) of each fragrance accord comprises less than 15 percent of the notes of said accord. The composition is particular useful for reed diffusors.

WO 2016 1196601 A1 refers to a fragrance composition comprising a fragrance compound and a solvent or carrier, wherein at least 10 percent by weight based on the total weight of solvent and carrier of the fragrance composition is one or more specific low vapor pressure volatile organic compound (VOC). The new fragrance composition is particularly well adapted to air care devices like for example reed diffusors.

GB 2474042 B2 claims a method of blending valerian oil with other essential oils plus water and a dispersing agent, using a reed diffuser to provide slow release of the composition properties into the environment for the purpose of helping to relieve stress and its associated symptoms in humans and other mammals. Preferably the composition contains valerian, vetivert, sweet basil and sage oils. The preferred dispersing agent is 3-methyl-3-methoxybutyl acetate.

Conventional liquid air fresheners include high levels of volatile organic compound (VOC) solvents such as such as for example dipropylene glycol methyl ether (Dowanol DPM), which are expensive and are disfavoured due to environmental and safety concern, and may raise regulatory concerns. Thus, many conventional air freshener formulations, including aqueous-based formulations, impose restrictions in fragrance creativity and limitations on the level of fragrance that can be employed due to fragrance solubility or solubilization issues. For example, perfumed liquid air fresheners containing high level of water, often require large amounts of solubilizers, such as surfactants, in order to achieve the desired clarity and stability of the final products. Such products may contain only low perfume contents, which causes a poor room impact in use or contain excess amounts of solubilizers with respect to higher perfume dosages, which have unsatisfactory impact on fragrance performance over time, such as fragrance release due to thereby accruing wick clogging. Further, conventional water-based air fresheners often suffer from certain disadvantages and limitations. Thus, the performance may be unsatisfactory due to limited or even unacceptable fragrance performance, product longevity, esthetical appearance, or temperature product stability over time.

In particular, reed diffuser compositions are typically based on expensive and sometimes ecologically harmful solvents such as for example dipropyleneglycol monomethyl ether (DPM). For this reason, perfume industry is looking for alternatives, preferably water-based systems. Water-based systems, however, mostly do not work technically or allow only low perfume contents. This causes a poor room impact in use. To get a similar fragrance performance for common systems, the same amount of perfume must be given to the water phase. To get this perfume content soluble it is necessary to increase the solubilizer content significantly. Unfortunately, with huge amounts of non-evaporating solubilizers the wick will be clogged after very short time in use and no further evaporation is possible.

The use of high levels of solubilizers can lead to unacceptable product performance, such as limited performance over time due to suppressed or poor fragrance release through delivery device such as wicks or reeds as a result of the solubilizer clogging the capillary channels in the diffuser. Therefore, there is a need for improved water-based air freshener formulations to address these and other problems.

The complex object of the present invention was to provide specific compositions for use in air fresheners devices, in particular wicks or reeds diffusers, solving the problems and disadvantages of the aqueous-based formulations mentioned above. Thus, within the context of the present invention, said compositions should be water-based formulations, which enhance the amount of addable perfume-oil with low solubilizer content and a combination of solvents in small amounts to optimize the solubility of the perfume reducing also the risk of solubilizer clogging the capillary channels in the diffuser, in particular of the wicks or reeds.

### DESCRIPTION OF THE INVENTION

A subject matter of the invention is a liquid air freshener system, comprising or consisting of:
(i) a container;
(ii) a liquid air freshener composition; and
(iii) a diffuser for diffusing the air freshener composition into surrounding air,
wherein the liquid air freshener composition is an oil-in-water emulsion comprising or consisting of:
(a) an aqueous phase;
(b) an oily phase comprising at least one perfume or aroma substance or perfume oil;
(c) at least one surfactant;
(d) at least one stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches and modified celluloses and mixtures thereof; and optionally
(e) at least one other additive and/or adjuvant;
wherein the amount of the surfactant is from 0.4 to 5% by weight, based on the total weight of the oily phase, the oil-in-water emulsion having an average oil droplet size of about 1 to about 15 μm.

Surprisingly, it was found that the use of an oil-in-water emulsion as defined above in a liquid air freshener system, in particular wicks or reeds diffusers, allows the enhancement of the amount of addable perfume or aroma substance as well as the reduction of the surfactant concentration in comparison to conventional aqueous-based freshener formulations, achieving a comparable or even an improved evaporation performance when compared to non-aqueous diffusible compositions based on dipropyleneglycol monomethyl ether.

Further, it was surprisingly found that typical clogging problems of the diffusers (wicks or reeds) associated with the high amounts of surfactants required in the conventional air freshener formulations, including aqueous-based formulations, have been remarkably reduced.

As mentioned above the oil-in-water emulsion having an average oil droplet size of about 1 to about 15 μm. It could be established that the average oil droplet size plays an important role in increasing the stability of the oil-in-water emulsion. In a particular preferred embodiment, the oil-in-water emulsion has an average oil droplet size ≥1 μm and <10 μm.

Oil droplet size can be measured by any known methods, such as by laser diffraction using known techniques and instruments. For example, a Mastersizer 2000 (Malvern Instruments of Malvern, Worchestershire, United Kingdom) can be used.

The component (a) of the oil-in-water emulsion of the liquid air freshener system according to the present invention is water or alternatively water in combination with a polar solvent, miscible in water but immiscible in the oily phase. Specific non-limiting examples of such water-miscible solvents include ethanol, DMSO (dimethyl sulfoxide), DPG (dipropylene glycol) or PEG (polyethylene glycol). Preferably, the aqueous phase of the oil-in-water emulsion of the present invention is pure water.

The proportion of the aqueous is from 25 to 99.9% by weight, preferably from 50 to 99% by weight, more preferred from 80 to 98% by weight, particularly preferred from 85 to 97% by weight, and most preferred from 89 to 96% by weight, relative to the total weight of the oil-in-water emulsion.

The component (b) of the oil-in-water emulsion of the liquid air freshener system according to the present invention is an oily phase comprising at least one perfume or aroma substance or perfume oil; eventually, in case of olfactory need, said at least one perfume or aroma substance or perfume oil can be mixed with DPM (Dipropylene glycol methyl ether) or ethanol.

In a preferred embodiment, the component (b) of the oil-in-water emulsion of the liquid air freshener system according to the present invention consists of the at least one perfume or aroma substance or perfume oil.

In a further preferred embodiment, the component (b) of the oil-in-water emulsion of the liquid air freshener system according to the present invention is free of dipropylene glycol methyl ether and/or ethanol.

The proportion of the component (b) is 0.1 to 75% by weight, preferably 1 to 50% by weight, more preferred 2 to 20% by weight, particularly preferred 2 to 10% by weight, and most preferred 3 to 8% by weight, relative to the total weight of the oil-in-water emulsion.

The mixing ratio of the component (a) to component (b) is preferably within a range of 999.09:0.01 to 250:750, preferably 990:10 to 500:500, more preferably 900:50 to 850:150.

The selection of at least one perfume or aroma substance or perfume oil of component (b) is not critical per se and is determined exclusively by the desired application. The only limiting factor is that these must either be present as oil or be sufficiently oil-soluble. Accordingly, the least one perfume or aroma substance of component (b) are hydrophobic or lipophilic substances.

In a preferred embodiment, the amount of at least one perfume or aroma substance or perfume oil in the oil-in-water emulsion of the liquid air freshener system according to the present invention is 0.1 to 75% by weight, preferably 1 to 50% by weight, more preferred 2 to 20% by weight, particularly preferred 2 to 10% by weight, and most preferred 3 to 8% by weight, relative to the total weight of the oil-in-water emulsion.

A perfume substance or aroma substance is understood here to mean a compound which is used for the primary purpose of conferring or modifying an odour. In other words, in order to be considered a perfuming substance, such an ingredient must be recognised by a person skilled in the art as being able to at least impart or modify the odour of a composition in a positive or pleasant way. For the purposes of the present invention, the terms “perfume oil” or “aroma” include a combination of perfuming ingredients for modifying or imparting an odour.

The following specified odoriferous or aroma substances can be used, either as individual substances or in mixtures with at least one other odoriferous or aroma substance, in a large number of fragrance or aroma mixtures, selected from an extensive range of natural and synthetic substances. However, the lists of these active substances and other ingredients are non-limiting and may include further active ingredients and other ingredients that are not elucidated further below.

Perfume substances or aroma substances which are advantageously suitable for combining are listed for example in S. Arctander, Perfume and Flavor Materials, volumes I and II, Montclair, N.J. 1969, private publication, and/or in H. Surburg, J. Panten, Common Fragrance and Flavor Materials, 6th edition, Wiley-VCFI, Weinheim 2016. The following list comprises examples of known odoriferous substances or aroma substances:
Fragrances and perfume oils may be natural odourant mixtures, such as those obtainable from plant sources, examples being extracts of natural raw materials such as essential oils, concretes, absolutes, resins, resinoids, balsams, tinctures such as for example: ambergris tincture; amyris oil; angelica seed oil; angelica root oil; anise oil; valerian oil; basil oil; tree moss absolute; bay oil; artemisia oil; benzoin resin; bergamot oil; beeswax absolute; birch tar oil; bitter almond oil; savory oil; buchu leaf oil; cabreuva oil; cade oil; calamus oil; camphor oil; cananga oil; cardamom oil; cascarilla oil; cassia oil; cassie absolute; castoreum absolute cedar leaf oil; cedarwood oil; cistus oil; citronella oil; lemon oil; copaiba balsam; copaiba balsam oil; coriander oil; costus root oil; cumin oil; cypress oil; davana oil; dill weed oil; dill seed oil; cau de brouts absolute; oak moss absolute; elemi oil; tarragon oil; eucalyptus citriodora oil; eucalyptus oil; fennel oil; pine-needle oil; galbanum oil; galbanum resin; geranium oil; grapefruit oil; guaiacwood oil; gurjun balsam; gurjun balsam oil; helichrysum absolute; helichrysum oil; ginger oil; iris root absolute; iris root oil; jasmine absolute; calamus oil; blue camomile oil; Roman camomile oil; carrot seed oil; cascarilla oil; pine-needle oil; spearmint oil; caraway oil; labdanum oil; labdanum absolute; labdanum resin; lavandin absolute; lavandin oil; lavender absolute; lavender oil; lemongrass oil; lovage oil; distilled lime oil; pressed lime oil; linaloe oil; Litsea cubeba oil; bay leaf oil; mace oil; marjoram oil; mandarin oil; massoia bark oil; mimosa absolute; ambrette oil; musk tincture; muscatel sage oil; nutmeg oil; myrrh absolute; myrrh oil; myrtle oil; clove leaf oil; clove bud oil; neroli oil; olibanum absolute; olibanum oil; opopanax oil; orange blossom absolute; orange oil; origanum oil; palmarosa oil; patchouli oil; perilla oil; Peru balsam oil; parsley leaf oil; parsley seed oil; petitgrain oil; peppermint oil; pepper oil; pimento oil; pine oil; pennyroyal oil; rose absolute; rosewood oil; rose oil; rosemary oil; Dalmatian sage oil; Spanish sage oil; sandalwood oil; celery seed oil; spike lavender oil; star anise oil; styrax oil; tagetes oil; fir needle oil; tea tree oil; terpentine oil; thyme oil; Tolu balsam; tonka absolute; tuberose absolute; vanilla extract; violet leaf absolute; verbena oil; vetiver oil; juniper berry oil; cognac oil; wormwood oil; wintergreen oil; ylang ylang oil; hyssop oil; civet absolute; cinnamon leaf oil; cinnamon bark oil, and fractions thereof or constituents isolated therefrom;
individual odoriferous substances from the group comprising hydrocarbons, such as for example 3-carene; alpha-pinene; beta-pinene; alpha-terpinene; gamma-terpinene; p-cymene; bisabolene; camphene; caryophyllene; cedrene; famesene; limonene; longifolene; myrcene; ocimene; valencene; (E,Z)-1,3,5-undecatriene; styrene; diphenylmethane;
aliphatic aldehydes and the acetals thereof such as for example hexanal; heptanal; octanal; nonanal; decanal; undecanal; dodecanal; tridecanal; 2-methyloctanal; 2-methylnonanal; (E)-2-hexenal; (Z)-4-heptenal; 2,6-dimethyl-5-heptenal; 10-undecenal; (E)-4-decenal; 2-dodecenal; 2,6,10-trimethyl-9-undecenal; 2,6,10-trimethyl-5,9-undecadienal; heptanal diethylacetal; 1,1-dimethoxy-2,2,5-trimethyl-4-hexene; citronellyloxyacetaldehyde; 1-(1-methoxypropoxy)-(E/Z)-3-hexene;
cycloaliphatic aldehydes such as for example 2,4-dimethyl-3-cyclohexene carbaldehyde; 2-methyl-4-(2,2,6-trimethyl-cyclohexen-1-yl)-2-butenal; 4-(4-hydroxy-4-methylpentyl)-3-cyclohexene carbaldehyde; 4-(4-methyl-3-penten-1-yl)-3-cyclohexene carbaldehyde; aromatic and araliphatic aldehydes such as for example: benzaldehyde; phenylacetaldehyde; 3-phenylpropanal; hydratropaldehyde; 4-methylbenzaldehyde; 4-methylphenylacetaldehyde; 3-(4-ethylphenyl)-2,2-dimethylpropanal; 2-methyl-3-(4-isopropylphenyl)propanal; 2-methyl-3-(4-tert-butylphenyl)propanal; 2-methyl-3-(4-isobutylphenyl)propanal; 3-(4-tert-butylphenyl)propanal; cinnamaldehyde; alpha-butylcinnamaldehyde; alpha-amylcinnamaldehyde; alpha-hexylcinnamaldehyde; 3-methyl-5-phenylpentanal; 4-methoxybenzaldehyde; 4-hydroxy-3-methoxybenz-aldehyde; 4-hydroxy-3-ethoxybenzaldehyde; 3,4-methylenedioxybenzaldehyde; 3,4-dimethoxybenzaldehyde; 2-methyl-3-(4-methoxyphenyl)propanal; 2-methyl-3-(4-methylenedioxyphenyl)propanal;
esters of aliphatic carboxylic acids such as for example (E)- and (Z)-3-hexenyl formate; ethyl acetoacetate; isoamyl acetate; hexyl acetate; 3,5,5-trimethylhexyl acetate; 3-methyl-2-butenyl acetate; (E)-2-hexenyl acetate; (E)- and (Z)-3-hexenyl acetate; octyl acetate; 3-octyl acetate; 1-octen-3-yl acetate; ethyl butyrate; butyl butyrate; isoamyl butyrate; hexyl butyrate; (E)- and (Z)-3-hexenyl-isobutyrate; hexyl crotonate; ethyl isovalerate; ethyl-2-methyl pentanoate; ethyl hexanoate; allyl hexanoate; ethyl heptanoate; allyl heptanoate; ethyl octanoate; ethyl-(E,Z)-2,4-decadienoate; methyl-2-octinate; methyl-2-noninate; allyl-2-isoamyloxyacetate; methyl-3, 7-dimethyl-2,6-octadienoate; 4-methyl-2-pentyl-crotonate;
esters of cyclic alcohols such as for example 2-tert-butylcyclohexyl acetate; 4-tert-butylcyclohexyl acetate; 2-tert-pentylcyclohexyl acetate; 4-tert-pentylcyclohexyl acetate; 3,3,5-trimethylcyclohexyl acetate; decahydro-2-naphthyl acetate; 2-cyclopentylcyclopentyl crotonate; 3-pentyltetrahydro-2H-pyran-4-yl acetate; decahydro-2,5,5,8a-tetramethyl-2-naphthyl acetate; 4,7-methano-3a,4,5,6,7,7a-hexahydro-5- or 6-indenyl acetate; 4,7-methano-3a,4,5,6,7,7a-hexahydro-5- or 6-indenyl propionate; 4,7-methano-3a,4,5,6,7,7a-hexahydro-5- or 6-indenyl isobutyrate; 4,7-methanooctahydro-5- or 6-indenyl acetate;
esters of araliphatic alcohols and aliphatic carboxylic acids such as for example benzyl acetate; benzyl propionate; benzyl isobutyrate; benzyl isovalerate; 2-phenylethyl acetate; 2-phenylethyl propionate; 2-phenylethyl isobutyrate; 2-phenylethyl isovalerate; 1-phenylethyl acetate; alpha-trichloromethylbenzyl acetate; alpha, alpha-dimethylphenylethyl acetate; alpha, alpha-dimethylphenyl-ethyl butyrate; cinnamyl acetate; 2-phenoxyethyl isobutyrate; 4-methoxybenzyl acetate;
esters of cycloaliphatic alcohols such as for example 1-cyclohexylethyl crotonate; esters of cycloaliphatic carboxylic acids such as for example allyl-3-cyclohexyl propionate; allylcyclohexyl oxyacetate; cis- and trans-methyl dihydrojasmonate; cis- and trans-methyl jasmonate; methyl-2-hexyl-3-oxocyclopentane carboxylate; ethyl-2-ethyl-6,6-dimethyl-2-cyclohexene carboxylate; ethyl-2,3,6,6-tetramethyl-2-cyclo-hexene carboxylate; ethyl-2-methyl-1,3-dioxolane 2-acetate;
aromatic and araliphatic carboxylic acids and the esters thereof such as for example: benzoic acid; phenylacetic acid; methyl benzoate; ethyl benzoate; hexyl benzoate; benzyl benzoate; methylphenyl acetate; ethylphenyl acetate; geranylphenyl acetate; phenylethylphenyl acetate; methyl cinnamate; ethyl cinnamate; benzyl cinnamate; phenylethyl cinnamate; cinnamyl cinnamate; allyl phenoxy acetate; methyl salicylate; isoamyl salicylate; hexyl salicylate; cyclohexyl salicylate; cis-3-hexenyl salicylate; benzyl salicylate; phenylethyl salicylate; methyl-2, 4-dihydroxy-3,6-dimethylbenzoate; ethyl-3-phenyl glycidate; ethyl-3-methyl-3-phenyl glycidate;
aliphatic alcohols such as for example hexanol; octanol; 3-octanol; 2,6-dimethylheptanol; 2-methyl-2-heptanol; 2-methyl-2-octanol; (E)-2-hexenol; (E)- and (Z)-3-hexenol; 1-octen-3-ol; mixtures of 3,4,5,6,6-pentamethyl-3,4-hepten-2-ol and 3,5,6,6-tetramethyl-4-methylencheptan-2-ol; (E,Z)-2,6-nonadienol; 3,7-dimethyl-7-methoxyoctan-2-ol; 9-decenol; 10-undecenol; 4-methyl-3-decen-5-ol;
aliphatic ketones and the oximes thereof such as for example 2-heptanone; 2-octanone; 3-octanone; 2-nonanone; 5-methyl-3-heptanone; 5-methyl-3-heptanone oxime; 2,4,4,7-tetramethyl-6-octen-3-one; 6-methyl-5-hepten-2-one;
aliphatic sulphur-containing compounds such as for example 3-methylthio-hexanol; 3-methylthiohexyl acetate; 3-mercaptohexanol; 3-mercaptohexyl acetate; 3-mercaptohexyl butyrate; 3-acetylthiohexyl acetate; 1-menthen-8-thiol;
aliphatic nitriles such as for example 2-nonenoic acid nitrile; 2-undecenoic acid nitrile; 2-tridecenoic acid nitrile; 3,12-tridecadienoic acid nitrile; 3,7-dimethyl-2,6-octadienoic acid nitrile; 3,7-dimethyl-6-octenoic acid nitrile;
acyclic terpene alcohols such as for example: citronellol; geraniol; nerol; linalool; lavandulol; nerolidol; farnesol; tetrahydrolinalool; tetrahydrogeraniol; 2,6-dimethyl-7-octen-2-ol; 2,6-dimethyloctan-2-ol; 2-methyl-6-methylene-7-octen-2-ol; 2,6-dimethyl-5,7-octadien-2-ol; 2,6-dimethyl-3,5-octadien-2-ol; 3,7-dimethyl-4,6-octadien-3-ol; 3,7-dimethyl-1,5,7-octatrien-3-ol; 2,6-dimethyl-2,5,7-octatrien-1-ol; and the formates, acetates, propionates, isobutyrates, butyrates, isovalerates, pentanoates, hexanoates, crotonates, tiglinates and 3-methyl-2-butenoates thereof;
acyclic terpene aldehydes and ketones such as for example geranial; neral; citronellal; 7-hydroxy-3,7-dimethyloctanal; 7-methoxy-3,7-dimethyloctanal; 2,6,10-trimethyl-9-undecenal; geranyl acetone; and the dimethyl and diethyl acetals of geranial, neral, 7-hydroxy-3,7-dimethyloctanal;
cyclic terpene alcohols such as for example: menthol; isopulegol; alpha-terpineol; terpinenol-4; menthan-8-ol; menthan-1-ol; menthan-7-ol; borneol; isoborneol; linalool oxide; nopol; cedrol; ambrinol; vetiverol; guaiol; and the formates, acetates, propionates, isobutyrates, butyrates, isovalerates, pentanoates, hexanoates, crotonates, tiglinates and 3-methyl-2-butenoates thereof;
cyclic terpene aldehydes and ketones such as for example menthone; isomenthone; 8-mercaptomenthan-3-one; carvone; camphor; fenchone; alpha-ionone; beta-ionone; alpha-n-methyl ionone; beta-n-methyl ionone; alpha-isomethyl ionone; beta-isomethyl ionone; alpha-irone; alpha-damascone; beta-damascone; beta-damascenone; delta-damascone; gamma-damascone; 1-(2,4,4-trimethyl-2-cyclohexen-1-yl)-2-buten-1-one; 1,3,4,6,7,8a-hexahydro-1,1,5,5-tetramethyl-2H-2,4a-methanonaphthalen-8 (5H)-one; 2-methyl-4-(2,6,6-trimethyl-1-cyclohexen-1-yl)-2-butenal; nootkatone; dihydronootkatone; 4,6,8-megastigmatrien-3-one; alpha-sinensal; beta-sinensal; acetylated cedarwood oil (methylcedryl ketone);
cyclic alcohols such as for example: 4-tert-butylcyclohexanol; 3,3,5-thmethylcyclohexanol; 3-isocamphylcyclohexanol; 2,6,9-trimethyl-Z2,Z5,E9-cyclododecatrien-1-ol; 2-isobutyl-4-methyltetrahydro-2H-pyran-4-ol;
cycloaliphatic alcohols such as for example alpha-3, 3-thmethylcyclohexylmethanol; 1-(4-isopropylcyclohexyl)ethanol; 2-methyl-4-(2,2,3-thmethyl-3-cyclopent-1-yl)butanol; 2-methyl-4-(2,2,3-trimethyl-3-cyclopent-1-yl)-2-buten-1-ol; 2-ethyl-4-(2,2,3-thmethyl-3-cyclopent-1-yl)-2-buten-1-ol; 3-methyl-5-(2,2,3-thmethyl-3-cyclopent-1-yl)-pentan-2-ol; 3-methyl-5-(2,2,3-trimethyl-3-cyclopent-1-yl)-4-penten-2-ol; 3,3-dimethyl-5-(2,2,3-trimethyl-3-cyclopent-1-yl)-4-penten-2-ol; 1-(2,2,6-trimethylcyclohexyl)pentan-3-ol; 1-(2,2,6-trimethylcyclohexyl)hexan-3-ol;
cyclic and cycloaliphatic ethers such as for example: cineole; cedryl methyl ether; cyclododecyl methyl ether; 1,1-dimethoxycyclododecane; (ethoxymethoxy)cyclododecane; alpha-cedrene epoxide; 3a, 6,6,9a-tetramethyldodecahydronaphtho[2,1-b]furan; 3a-ethyl-6,6,9a-trimethyldodeca-hydronaphtho[2,1-b]furan; 1,5,9-trimethyl-13-oxabicyclo[10.1 0]trideca-4,8-diene; rose oxide; 2-(2,4-dimethyl-3-cyclohexen-1-yl)-5-methyl-5-(1-methylpropyl)-1,3-dioxane;
cyclic and macrocyclic ketones such as for example 4-tert-butylcyclohexanone; 2,2,5-trimethyl-5-pentylcyclopentanone; 2-heptylcyclopentanone; 2-pentylcyclo-pentanone; 2-hydroxy-3-methyl-2-cyclopenten-1-one; 3-methyl-cis-2-penten-1-yl-2-cyclopenten-1-one; 3-methyl-2-pentyl-2-cyclopenten-1-one; 3-methyl-4-cyclopentadecenone; 3-methyl-5-cyclopentadecenone; 3-methylcyclopentadecanone; 4-(1-ethoxyvinyl)-3,3,5,5-tetramethylcyclohexanone; 4-tert-pentylcyclohexanone; 5-cyclohexadecen-1-one; 6,7-dihydro-1,1,2,3,3-pentamethyl-4(5H)-indanone; 8-cyclohexadecen-1-one; 9-cycloheptadecen-1-one; cyclopentadecanone; cyclohexadecanone;
cycloaliphatic ketones such as for example 1-(3,3-dimethyl-cyclohexyl)-4-penten-1-one; 2,2-dimethyl-1-(2,4-dimethyl-3-cyclohexene-1-yl)-1-propanone; 1-(5,5-dimethyl-1-cyclohexen-1-yl)-4-penten-1-one; 2,3,8,8-tetramethyl-1,2,3,4,5,6,7,8-octahydro-2-naphthalenyl methyl ketone; methyl-2,6,10-trimethyl-2,5,9-cyclododecatrienyl ketone; tert-butyl-(2,4-dimethyl-3-cyclohexen-1-yl)ketone;
aromatic hydrocarbons such as for example styrol and diphenyl methane;
araliphatic alcohols such as for example benzyl alcohol; 1-phenylethyl alcohol; 2-phenylethyl alcohol; 3-phenylpropanol; 2-phenylpropanol; 2-phenoxyethanol; 2,2-dimethyl-3-phenylpropanol; 2,2-dimethyl-3-(3-methylphenyl)propanol; 1,1-dimethyl-2-phenylethyl alcohol; 1, 1-dimethyl-3-phenylpropanol; 1-ethyl-1-methyl-3-phenylpropanol; 2-methyl-5-phenylpentanol; 3-methyl-5-phenylpentanol; 3-phenyl-2-propen-1-ol; 4-methoxybenzyl alcohol; 1-(4-isopropylphenyl)ethanol;
araliphatic ethers such as for example: 2-phenyl ethyl methyl ether; 2-phenyl ethyl isoamyl ether; 2-phenyl ethyl 1-ethoxyethyl ether; phenylacetaldehyde dimethylacetal; phenylacetaldehyde diethylacctal; hydratropaldehyde dimethylacetal; phenylacetaldehyde glycerol acetal; 2,4,6-trimethyl-4-phenyl-1,3-dioxane; 4, 4a, 5,9b-tetrahydroindeno[1,2-d]-m-dioxin; 4,4a,5,9b-tetrahydro-2,4-dimethylindeno[1,2-d]-m-dioxin;
aromatic and araliphatic ketones such as for example: acetophenone; 4-methyl-acetophenone; 4-methoxyacetophenone; 4-tert-butyl-2,6-dimethylacetophenone;
4-phenyl-2-butanone; 4-(4-hydroxyphenyl)-2-butanone; 1-(2-naphthalenyl)ethanone; 2-benzofuranylethanone; (3-methyl-2-benzofuranyl)ethanone; benzophenone; 1,1,2,3,3,6-hexamethyl-5-indanyl methyl ketone; 6-tert-butyl-1,1-dimethyl-4-indanyl methyl ketone; 1-[2,3-dihydro-1,1,2,6-tetramethyl-3-(1-methylethyl)-1H-5-indenyl]ethanone; 5′,6′,7′,8′-tetrahydro-3′,5′,5′,6′,8′,8′-hexamethyl-2-acetonaphthone; nitrogenous aromatic compounds such as for example: 2,4,6-trinitro-1,3-dimethyl-5-tert-butylbenzene; 3,5-dinitro-2,6-dimethyl-4-tert-butyl aceto-phenone; cinnamonitrile; 3-methyl-5-phenyl-2-pentenoic acid nitrile; 3-methyl-5-phenylpentanoic acid nitrile; methyl anthranilate; methyl-N-methyl anthranilate; Schiff bases of methyl anthranilate with 7-hydroxy-3,7-dimethyloctanal, 2-methyl-3-(4-tert-butylphenyl)propanal or 2,4-dimethyl-3-cyclohexene carbaldehyde 6-isopropyl quinoline; 6-isobutyl quinoline; 6-sec-butyl quinoline; 2-(3-phenylpropyl)pyridine; indole; skatole; 2-methoxy-3-isopropylpyrazine; 2-isobutyl-3-methoxypyrazine;
phenols, phenyl ethers and phenyl esters such as for example: estragole; anethole; eugenol; eugenyl methyl ether; isoeugenol; isoeugenyl methyl ether; thymol; carvacrol; diphenyl ether; beta-naphthyl methyl ether; beta-naphthyl ethyl ether; beta-naphthyl isobutyl ether; 1,4-dimethoxybenzene; eugenyl acetate; 2-methoxy-4-methylphenol; 2-ethoxy-5-(1-propenyl)phenol; p-cresyl phenyl acetate;
heterocyclic compounds such as for example: 2,5-dimethyl-4-hydroxy-2H-furan-3-one; 2-ethyl-4-hydroxy-5-methyl-2H-furan-3-one; 3-hydroxy-2-methyl-4H-pyran-4-one; 2-ethyl-3-hydroxy-4H-pyran-4-one;
lactones such as for example: 1,4-octanolide; 3-methyl-1,4-octanolide; 1.4-nonanolide; 1,4-decanolide; 8-decen-1,4-olide; 1,4-undecanolide; 1,4-dodecan-olide; 1,5-decanolide; 1,5-dodecanolide; 4-methyl-1,4-decanolide; 1,15-penta-decanolide; cis- and trans-11-pentadecen-1,15-olide; cis- and trans-12-pentadecen-1,15-olide; 1,16- hexadecanolide; 9-hexadecen-1, 16-olide; 10-oxa-1, 16-hexadecanolide; 11-oxa-1, 16-hexadecanolide; 12-oxa-1,16-hexadecanolide; ethylene 1,12-dodecanedioate; ethylene 1,13-tridecanedioate; coumarin; 2,3-dihydrocoumarin; octahydrocoumarin;
and mixtures of the above substances.

The oil-in-water emulsion of the liquid air freshener system according to the present invention also comprises at least one surfactant or emulsifying agent. The choice of said at least one surfactant has a significant impact on the characteristics of the emulsion, especially in order to retain the stability of the emulsion.

Surfactants are classified by their hydrophile-lipophile balance (HLB) number, used as a measure of the ratio of these groups and useful in identifying surfactants for oil-and-water emulsification. It is a value between 0 to 20 which defines the affinity of a surfactant for water or oil. HLB numbers are calculated for non-ionic surfactants, and these surfactants have numbers ranging from 0 to 20. HLB numbers over 10 indicate an affinity for water (hydrophilia), and HLB numbers under 10 indicate an affinity for oil (lipophilia). In order to form and stabilise the oil-in-water macroemulsion according to the present invention, at least one surfactant with an HLB number of between 8 and 18 is used. Preferably, a surfactant with an HLB number between 10 and 14 is used. In a most preferred variant, the macroemulsion of the present invention comprises at least one surfactant with an HLB number of between 11 and 13.

The at least one surfactant or emulsifying agent to be used in the oil-in-water emulsion of the liquid air freshener system according to the present invention is selected from the group consisting of non-ionic surfactants, anionic surfactants, and amphoteric surfactants and mixtures thereof.

Typical examples of non-ionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed eithers or mixed formals, optionally partially oxidized alk(en)yl oligoglycosides or glucuronic acid derivatives, fatty acid-N-alkyl glucamides, protein hydrolysates (in particular wheat-based plant products), polyol fatty acid esters, sucrose esters, sorbitan esters, polysorbates and aminoxides.

Typical anionic surfactants or emulsifiers are aliphatic fatty acids with 12 to 22 carbon atoms, such as e.g. palmitic acid, stearic acid or behenic acid, and dicarboxylic acids with 12 to 22 carbon atoms, such as e.g. azelaic acid or sebacic acid. Typical examples of anionic surfactants are soaps, alkylbenzene sulfonates, alkane sulfonates, olefin sulfonates, alkyl ether sulfonates, glyceryl ether sulfonates, a-methyl ester sulfonates, sulfofatty acids, alkyl sulfates, alkyl ether sulfates, glyceryl ether sulfates, fatty acid ether sulfates, hydroxy mixed ether sulfates, monoglyceride (ether) sulfates, fatty acid amide (ether) sulfates, mono- and dialkylsulfosuccinates, mono- and dialkylsulfosuccinamates, sulfotriglycerides, amide soaps, ether carboxylic acids and salts thereof, fatty acid isethionates, fatty acid sarcosinates, fatty acid taurides, N-acylamino acids, such as e.g. acyl lactylates, acyl tartrates, acyl glutamates and acyl aspartates, alkyl oligoglycoside sulfates, protein fatty acid condensates (in particular wheat-based plant products) and alkyl(ether) phosphates.

Moreover, zwitterionic surfactants may be used as surfactants or emulsifiers. Surfactant compounds having in the molecule at least one quaternary ammonium group and at least one carboxylate and one sulfonate group are referred to as zwitterionic surfactants. Particularly suitable zwitterionic surfactants are the so-called betaines, such as the N-alkyl-N,N-dimethyl ammonium glycinates, e.g. coconut alkyldimethyl ammonium glycinate, the N-acylaminopropyl-N,N-dimethyl ammonium glycinates, for example coconut acylaminopropyl dimethyl ammonium glycinate, 2-alkyl-3-carboxylmethyl-3-hydroxyethylimidazolines with 8 to 18 C atoms respectively in the alkyl or acyl group, and coconut acylaminoethyl hydroxyethyl carboxymethyl glycinate. Particularly preferred is the fatty acid amide derivative known by the CTFA name cocamidopropyl betaine. Further suitable emulsifiers are ampholytic surfactants. Ampholytic surfactants are understood to refer to surfactant compounds which, in addition to one C8/18 alkyl or acyl group in the molecule, comprise at least one free amino group and at least one —COOH— or —SO₃H group and are capable of forming inner salts. Examples of suitable ampholytic surfactants arc N-alkylglycines, N-alkylpropionic acids, N-alkylaminobutyric acids, N-alkyliminodipropionic acids, N-hydroxyethyl-N-alkylamidopropylglycines, N-alkyltaurines, N-alkylsarcosines, 2-alkylaminopropionic acids and alkylaminoacetic acids with approximately 8 to 18 C atoms respectively in the alkyl group. Particularly preferred ampholytic surfactants are N-coconut alkylaminopropionate, coconut acylaminoethyl aminopropionate and C12/18 acylsarcosine. Typical examples of amphoteric or zwitterionic surfactants are alkyl betaine, alkyl amidobetaines, aminopropionates, aminoglycinates, imidazolinium betaines and sulfobetaines. The above-mentioned surfactants are exclusively known compounds. Typical further examples of particularly suitable surfactants are fatty alcohol polyglycol ether sulfates, monoglyceride sulfates, mono- and/or dialkylsulfosuccinates, fatty acid isethionates, fatty acid sarcosinates, fatty acid taurides, fatty acid glutamates, a-olefin sulfonates, ether carboxylic acids, alkyl oligoglycosides, fatty acid glucamides, alkyl amido betaines, amphoacetals and/or protein fatty acid condensates, the latter preferably based on wheat proteins.

Preferably, the at least one non-ionic surfactant is selected from the group consisting of:

| PN/CAS | | |
|---|---|---|
| non-ionic | Name | Chemical composition |
| 358712 | SymSol PF-3 | water (aqua), pentylene glycol, |
| | | sodium lauryl sulphoacetate, |
| | | sodium oleoyl sarcosinate, |
| | | sodium chloride, |
| | | disodium sulphoacetate, |
| | | sodium oleate, |
| | | sodium sulphate |
| 3332-27-2 | Ammonyx MO | N,N-dimethyldodecylamine-N-oxide, |
| | | lauryldimethylamine-N-oxide |
| 68551-12-2 | Berol 175 | laureth-8, Empilan KB-8, Walloxen LM |
| | | 80, Elfapur LM 75 S, |
| 68439-46-3 | Berol 266 | C9-11 PARETH-5,5 |
| 26468-86-0 | Berol 840 | 2-ethylhexanol ethoxylat |
| | Empilan KB 10 | ethoxylated C10-C16 alcohols (10 EO) |
| 68131-39-5 | Empilan KCL 7 | ethoxylated C12-C15 alcohols, (7 EO) |
| | Emulan EL | ethoxylated Ricinus oil (45 EO) |
| | emulsifier | hydrogenated, ethoxylated castor oil |
| | Cremophor CO 455 | (40 EO) |
| 61791-12-6 | emulsifier | hydrogenated, ethoxylated castor oil |
| | Cremophor EL | (35 EO) |
| 9005-64-5 | emulsifier Tween 20 | sorbitan ester polyethylene glycol ether |
| | | (20 EO) |
| 69011-36-5 | emulsifier Trideceth-9 | poly(oxy-1,2-ethanedily), |
| | | branched alpha-tridecyl-omega-hydroxy |
| 61791-12-6 | FINDET ® AR/52 | PEG-40 castor oil |
| 68515-73-1 | Glucopon ® 215 UP | capryl glucoside |
| | Glucopon ® 50 G | alkylpolyglucoside, silicate, sulphate |
| 110615-47-9 | Glucopon ® 600 CSUP | C10-C16 alkylpolyglucoside |
| 110615-47-9 | Glucopon ® 650 EC | coco glucoside |
| 69011-36-5 | Hoesch T 9 90% | Trideceth-9 |
| 68439-49-6 | Hoesch TG 20 | ceteareth-20 |
| 26183-52-8 | Imbentin AG/100/080 | C10 oxoalcohol ethoxylate (8 EO) |
| 68439-49-6 | Imbentin AG/168S/500 G | ceteareth-50 |
| 68131-39-5 | Imbentin C/125/030 | C12-C15 oxoalcohol ethoxylate (3 EO) |
| R-FRA00145 | Lutensol A 8 | C12-C14 oxoalcohol (8 EO) |
| R-FRA00166 | Lutensol XP 140 | isodecanol ethoxylate (14 EO) |
| 69011-36-5 | Marlipal O 13/70 | Trideceth-7 |
| 110615-47-9 | Plantacare 1200 UP | lauryl glycoside |
| 141464-42-8 | Plantacare 818 UP | C18-C16 fatty alcohol glucoside |
| R-FRA00069 | Plurafac LF 403 | EO/PO oxoalcohol |
| 68140-00-1 | Rewomid C 212 | coco fatty acid monoethanolamide |
| 69011-36-5 | solubilizer | PEG-40 hydrogenated castor oil, |
| | | Trideceth-9, propylene glycol, water |
| 2605-79-0 | Tegotens DO | decyldimethylaminoxide |
| R-FRA00058 | Tween 80 V Pharma | polyoxyethylene sorbitan fatty acid ester |

Preferably, the at least one anionic surfactants is selected from the group consisting of:

| PN/CAS | | |
|---|---|---|
| Anionic | Name | Chemical composition |
| 14960-06-6 | Deriphat 160 C | sodium lauriminodipropionate |
| 151-21-3 | dodecyl sulphate | dodecyl sulphate, sodium salt |
| | sodium salt | |
| | Empicol EGC 70 | magnesium laureth sulphate |
| 68891-38-3 | Hansanol NS 242 conc | sodium laureth sulphate |
| | Hoesch AE 50 | sodium dodecyl benzene |
| | | sulphonate |
| 85711-69-9 | Hoesch NAS 60 | C13-C17 sec-alkane sulphonate, |
| | | sodium salt |
| 28348-53-0 | Hoesch NCS | sodium isopropyl benzol |
| | | sulphonate |
| 119415-05-3 | Hostaphat KW 340 D | triceteareth-4 phosphate |
| 584-08-7 | Hostapur OSB | olefin sulphonate, sodium salt |
| 68411-30-3 | Marlon A 330 | sodium dodecylbenzene |
| | | sulphonate |
| 12068-03-0 | Marlon ARL | benzolsulphonic acid, |
| | | C10-C13 alkyl derivates, |
| | | sodium salt, sodium |
| | | toluenesulphonate |
| 25155-30-0 | Nacconol 90 G | linear sodium alkylbenzene |
| | | sulphonate |
| 110615-47-9 | Plantacare PS 10 | sodium lauryl sulphate, |
| | | lauryl glucoside |
| | Texapon 842 | sodium octylsulphate (C8) |
| 151-21-3 | Texapon K 12 Powder | sodium C12 fatty alcohol sulphate, |
| | | sodium lauryl sulphate |
| 78330-30-0 | Zetesol AO 328 U | sodium pareth sulphate, |
| | | ethoxylated C13-C15 alcohol |
| 68439-50-9 | Zetesol NL-2 | C12-C14 sodium lauryl ether |
| | | sulphate |

Preferably, the at least one amphoteric surfactant is selected from the group consisting of:

| PN/CAS | | |
|---|---|---|
| Amphoteric | Name | Chemical composition |
| | Akypo LM-40 | alkyl ether carboxylic acid |
| 68955-55-5 | Ammonyx LO | lauryl myristyl dimethylaminoxide |
| 68140-00-1 | Comperlan 100 | C12-C18 coconut fatty acid |
| | | monoethanolamide |
| 68424-94-2 | Dehyton AB 30 | coco dimethylammonium betaine |
| 61789-40-0 | Dehyton K | cocoyl amide propyldimethyl glycine |
| 26635-93-8 | Genamin O-020 | ethoxylated oleyl amine (2 EO) |
| 61788-90-7 | Genaminox ® CSL | alkyl dimethylaminoxide |
| 61789-40-0 | Hoesch Betain 40 | cocoamidopropyl betaine |

where EO is ethylene oxide and PO is propylene oxide.

In a preferred embodiment, the at least one surfactant is a non-ionic surfactant or emulsifier. Non-limiting examples of non-ionic surfactants which may be cited include those belonging to the classes of:
C_{12/18} fatty acid monoesters and diesters of addition products of 1 to 30 mol ethylene oxide onto glycerol;
glycerol mono- and diesters and sorbitan mono- and diesters of saturated and unsaturated fatty acids containing 6 to 22 carbon atoms and ethylene oxide addition products thereof;
polyol esters and, in particular, polyglycerol esters such as, for example, polyglycerol polyricinoleate, polyglycerol poly-12-hydroxystearate or polyglycerol dimerate isostearate. Mixtures of compounds from several of these classes are also suitable;
partial esters based on linear, branched, unsaturated or saturated C_{6/22} fatty acids, ricinoleic acid and 12-hydroxystearic acid and glycerol, polyglycerol, pentaerythritol, dipentaerythritol, sugar alcohols (for example sorbitol), alkyl glucosides (for example methyl glucoside, butyl glucoside, lauryl glucoside) and polyglucosides (for example cellulose)
addition products of 2 to 30 mol of ethylene oxide and/or 0 to 5 mol of propylene oxide to linear fatty alcohols with 8 to 22 C atoms, fatty acids with 12 to 22 C atoms, alkylphenols with 8 to 15 C atoms in the alkyl group and alkylamines with 8 to 22 carbon atoms in the alkyl radical;
alkyl and/or alkenyl oligoglycosides with 8 to 22 carbon atoms in the alk(en)yl radical and ethoxylated analogs thereof;
addition products of 1 to 15 mol of ethylene oxide to castor oil and/or hardened castor oil;
addition products of 15 to 60 mol of ethylene oxide to castor oil and/or hydrogenated castor oil;
partial esters of glycerol and/or sorbitan with unsaturated, linear or saturated, branched fatty acids with 12 to 22 carbon atoms and/or hydroxycarboxylic acids with 3 to 18 carbon atoms and adducts thereof with 1 to 30 mol of ethylene oxide;
partial esters of polyglycerol (average degree of autocondensation 2 to 8), polyethylene glycol (molecular weight 400 to 5000 g/mol), trimethylolpropane, pentaerythritol, sugar alcohols (e.g. sorbitol), alkyl glycosides (e.g. methyl glycoside, butyl glycoside, lauryl glycoside) and polyglycosides (e.g. cellulose) with saturated and/or unsaturated, linear or branched fatty acids with 12 to 22 carbon atoms and/or hydroxycarboxylic acids with 3 to 18 carbon atoms and adducts thereof with 1 to 30 mol of ethylene oxide;
mixed esters of pentaerythritol, fatty acids, citric acid and fatty alcohol and/or mixed esters of fatty acids with 6 to 22 carbon atoms, methylglucose and polyols, preferably glycerol or polyglycerol, mono, di- and trialkyl phosphate and mono, di- and/or tri-PEG alkyl phosphate and salts thereof;
alkyl and/or alkenyl oligoglycosides;
lanolin alcohols;
polysiloxane-polyalkyl-polyether copolymers or corresponding derivatives; block copolymers, e.g. PEG-30 dipolyhydroxy stearate;
polymer emulsifiers, e.g. of the Pemulen type (TR-1, TR-2) from Goodrich or
Cosmedia® SP from Cognis;
polyalkylene glycols and glycerol carbonate.

In the following, particularly suitable emulsifiers are described in further detail:
Alkoxylates. The addition products of ethylene oxide and/or propylene oxide to fatty alcohols, fatty acids, alkylphenols or castor oil constitute known, commercially obtainable products. These are homolog mixtures whose average degree of alkoxylation corresponds to the ratio of the amount of substance of ethylene oxide and/or propylene oxide to the substrates with which the addition reaction is carried out. C12/18 fatty acid mono-and-diesters of addition products of ethylene oxide to glycerol are known as refatting agents for cosmetic preparations.
Alkyl and/or alkenyl oligoglycosides. Alkyl and/or alkenyl oligoglycosides and the production and use thereof are known from the prior art. In particular, they are produced by reacting glucose or oligosaccharides with primary alcohols with 8 to 18 carbon atoms. With respect to the glycoside radical, both monoglycosides in which a cyclic sugar radical is glycosidically bound to the fatty alcohol and oligomeric glycosides with a preferred degree of oligomerization of approximately 8 are suitable. Here, the degree of oligomerization is a statistical mean value on which a homologous distribution that is common for such technical products is based.
Partial glycerides. Typical examples of suitable partial glycerides are hydroxystearic acid monoglyceride, hydroxystearic acid diglyceride, isostearic acid monoglyceride, isostearic acid diglyceride, oleic acid monoglyceride, oleic acid diglyceride, ricinoleic acid monoglyceride, ricinoleic acid diglyceride, linoleic acid monoglyceride, linoleic acid diglyceride, linolenic acid monoglyceride, linolenic acid diglyceride, erucic acid monoglyceride, erucic acid diglyceride, tartaric acid monoglyceride, tartaric acid diglyceride, citric acid monoglyceride, citric acid diglyceride, malic acid monoglyceride, malic acid diglyceride and technical mixtures thereof, which may also secondarily comprise small amounts of triglyceride from the production process. Also suitable are addition products of 1 to 30, preferably 5 to 10 mol of ethylene oxide to the above-mentioned partial glycerides.
Sorbitan esters. Suitable sorbitan esters include sorbitan mono isostearate, sorbitan sesquiisostearate, sorbitan diisostearate, sorbitan triisostearate, sorbitan monooleate, sorbitan sesquioleate, sorbitan dioleate, sorbitan trioleate, sorbitan monoerucate, sorbitan sesquierucate, sorbitan dierucate, sorbitan trierucate, sorbitan monoricinoleate, sorbitan sesquiricinoleate, sorbitan diricinoleate, sorbitan triricinoleate, sorbitan monohydroxystearate, sorbitan sesquihydroxystearate, sorbitan dihydroxystearate, sorbitan trihydroxystearate, sorbitan monotartrate, sorbitan sesquitartrate, sorbitan ditartrate, sorbitan tritartrate, sorbitan monocitrate, sorbitan sesquicitrate, sorbitan dicitrate, sorbitan tricitrate, sorbitan monomaleate, sorbitan sesquimaleate, sorbitan dimaleate, sorbitan trimaleate and technical mixtures thereof. Also suitable are addition products of 1 to 30, preferably 5 to 10 mol of ethylene oxide to the above-mentioned sorbitan esters.
Polyglycerol esters. Typical examples of suitable polyglycerol esters are polyglyceryl-2 dipolyhydroxystearate (Dehymuls® PGPH), polyglycerol-3-diisostearate (Lameform® TGI), polyglyceryl-4 isostearate (Isolan® Gl 34), polyglyceryl-3 oleate, diisostearoyl polyglyceryl-3 diisostearate (Isolan® PDI), polyglyceryl-3 methylglucose distearate (Tego Care® 450), polyglyceryl-3 beeswax (Cera Beilina®), polyglyceryl-4 caprate (polyglycerol caprate T2010/90), polyglyceryl-3 cetyl ether (Chimexane® NL), polyglyceryl-3 distearate (Cremophor® GS 32) and polyglyceryl polyricinoleate (Admul® WOL 1403), polyglyceryl dimerate isostearate, as well as mixtures thereof. Examples of further suitable polyol esters are mono, di- and tri-esters of trimethylol propane or pentaerythritol, optionally reacted with 1 to 30 mol of ethylene oxide, with lauric acid, coconut fatty acid, tallow fatty acid, palmitic acid, stearic acid, oleic acid, behenic acid and the like.

The above surfactants can be used either individually or in combinations of two, three, four or even more surfactants, preferably in a mixture of two or three surfactants. The selection of such a surfactant system depends on the structure and polarity of the of the substances of the component (b), to be emulsified and to the remaining components of the oily phase of the macroemulsion. In a preferred variant of the present invention, a surfactant composition which is a combination of an ionic surfactant and a non-ionic surfactant, i.e. a combination of surfactants with different HLB values, is used for the preparation and stabilisation of the oil-in-water emulsion. For example, a surfactant having an HLB value of 19 can be combined with a surfactant having an HLB value of 2. The ionic surfactant provides the surface of the droplets with a charge. The resulting electrostatic charge prevents the droplets from coalescence. The non-ionic surfactant however prevents diffusion on the interface. The ratio of the ionic surfactant to the non-ionic surfactant, and, thus, the resulting HLB value, is adjusted to the final emulsion system. The oil-in-water emulsion of the air freshener system according to the present invention can best be stabilised by a surfactant composition which exhibits an appropriately adapted HLB number. In another preferred variant, therefore, the oil-in-water emulsion of the air freshener system according to the present invention comprises a surfactant composition having a final or mean HLB number between 10 and 14, wherein a final or mean HLB number between 11 and 13 stabilises the emulsion system best.

In a preferred embodiment, the oil-in-water emulsion of the liquid air freshener system according to the present invention comprises from 0.4 to 25% by weight of surfactant or emulsifying agent, preferably from 1 to 20% by weight, more preferably 4 to 15% by weight and most preferably from 8 to 12% by weight, based on the total weight of the oily or dispersed phase or from 0.004 to 18% by weight of surfactant or emulsifying agent, preferably from 0.01 to 10% by weight, more preferably from 0.08 to 3% by weight, particularly preferred from 0.2 to 1.5% by weight, and most preferred from 0.3 to 1.3% by weight, based on the total weight of the macroemulsion.

In a further preferred embodiment the ratio between surfactant or emulsifying agent at least one perfume or aroma substance or perfume oil is from 0.4:99.6 to 25:75, preferably from 1:99 to 20 to 80, more preferably from 4:96 to 15:85, and most preferred from 8:92 to 14:86.

The oil-in-water emulsion of the liquid air freshener system according to the present invention additionally comprises at least one stabiliser. Due to the low surfactant concentration, the stabiliser is an essential component, since it stabilises the emulsion and prevents it from degrading into phase separation (creaming, sedimentation), Ostwald ripening, aggregation processes (flocculation, coagulation, coalescence) or phase inversion. The stabiliser used in accordance with the present invention serves as a thickening agent or thickener and imbues the macroemulsion with a favourable rheological profile. A thickening agent or thickener is a substance which can increase the viscosity of a liquid without substantially changing its other properties. The stability of a emulsion depends directly on its viscosity. The higher the viscosity, the greater the stability of the macroemulsion. By using a stabiliser, i.e. a thickener, the migration velocity of the oil droplets in the macroemulsion is reduced and, thus, all emulsion breaking processes are decelerated. The emulsion of the present invention is thus kinetically stabilised. As mentioned above, the at least one stabiliser is selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches and modified celluloses and mixtures thereof.

In a preferred embodiment, the oil-in-water emulsion of the liquid air freshener system according to the present invention comprises up to 1.5% by weight of stabiliser, i.e. thickener, preferably in the range of 0.1 to 1.3% by weight and more preferably in the range of 0.6 to 1.1% by weight, based on the total amount of the macroemulsion.

In addition to the components described above, the oil-in-water emulsion of the liquid air freshener system according to the present invention can also optionally comprise additives and/or adjuvants. Preferably, the additives and/or adjuvants are selected from the group consisting of colorants, preservatives, deposition aids, etc., without being limited to such components.

In a preferred embodiment, the oil-in-water emulsion of the liquid air freshener system according to the present invention comprises up to 1% by weight, in particular 0.1 to 0.9% by weight of additives and/or adjuvants.

In a particularly preferred embodiment, the oil-in-water emulsion of the liquid air freshener system according to the present invention comprises or consists of the following components:
25 to 99.9% by weight, preferably 50 to 99% by weight, more preferably 80 to 98% by weight, particular preferred 81 to 97% by weightmost preferably 89 to 96% by weight, of the aqueous phase;
0.1 to 75% by weight, preferably 1 to 50% by weight, more preferably 2 to 20% by weight, particular preferred 3 to 10% by weight, most preferably 3 to 8% by weight, of the oily phase comprising at least one perfume or aroma substance or perfume oil;
0.004 to 18% by weight, preferably 0.01 to 10% by weight, more preferably 0.8 to 3% by weight, particularly preferred 0.2 to 1.5% by weight, most preferably 0.3 to 1.3% by weight of surfactant;
up to 1.5% by weight, preferably 0.6 to 1.1% by weight of stabiliser; and optionally
up to 1% by weight, preferably 0.1 to 0.9% by weight of additives and/or adjuvants;
based on the total weight of the macroemulsion.

In a further preferred embodiment, the diffuser is a natural or synthetic porous material that allows the diffusion of the air freshener composition. In certain embodiments, the diffuser comprises one or more wicks, reeds, or natural or synthetic porous diffusers. Preferably, the diffuser is selected from the group consisting of a wick, a reed diffuser, wooden or card board.

In a further preferred embodiment, the reed diffuser is made from a material selected from the group consisting of wood, fiber or paper.

In a further preferred embodiment, the reed diffuser is made from natural wood, preferably from softwood.

In certain embodiments, the air freshener further comprises removable means for preventing diffusion of the air freshener composition into surrounding air (e.g., a lid, cap, or other removable closure for the container). Said removable means can preventing diffusion of the liquid composition prior to the activation of the air freshener system, but can be opened or removed to permit diffusion of the liquid composition upon activation of the air freshener system.

In a further preferred embodiment, the liquid air freshener system according to the present invention is an electrical liquid air freshener.

The present invention concerns further to the use of an oil-in-water emulsion as a liquid air freshener composition in a liquid air freshener system, the oil-in-water emulsion comprising or consisting of:
(a) an aqueous phase;
(b) an oily phase comprising at least one perfume or aroma substance or perfume oil;
(c) at least one surfactant;
(d) at least one stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches and modified celluloses and mixtures thereof; and optionally
(e) at least one other additive and/or adjuvant;
wherein the amount of the surfactant is from 0.4 to 5% by weight, based on the total weight of the oily phase, and the oil-in-water emulsion having an average oil droplet size of about 1 to about 15 μm.

In this context, the above statements regarding the emulsion (for example components (a) to (e) . . . , etc.) and the liquid air freshener system also apply.

The liquid air freshener system is preferably a wick device or a reed diffuser.

The present invention concerns further to an oil-in-water emulsion comprising or consisting of:
(a) an aqueous phase;
(b) an oily phase comprising at least one perfume or aroma substance or perfume oil;
(c) at least one surfactant;
(d) at least one stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches and modified celluloses and mixtures thereof; and optionally
(e) at least one other additive and/or adjuvant;
wherein the amount of the surfactant is from 0.4 to 5% by weight, based on the total weight of the oily phase, the oil-in-water emulsion having an average oil droplet size ≥1 μm and <15 μm.

In this context, the above statements regarding the emulsion (for example components (a) to (e) . . . , etc.) also apply.

The present invention concerns further to the use of said oil-in-water emulsion having an average oil droplet size ≥1 μm and <15 μm as a liquid air freshener composition in a liquid air freshener system.

The oil-in-water emulsions according to the present invention can be produced by emulsifying methods using typical techniques which are well known in the prior art in the field of emulsions. In particular the oil-in-water emulsions described herein have been prepared according to the method described in WO 2020/234196 A1.

The present invention concerns further to a method for perfuming and/or refreshing, ambient air, the method comprising diffusing a liquid air freshener composition as described herein claims with the device as described herein.

### EXAMPLES

The following examples further illustrate the invention but are not to be construed as limiting its scope.

### Example 1

In order to evaluate the efficacy of a liquid air freshener system according to the present invention evaporation tests have been conducted, wherein different liquid air freshener compositions have been used.

In a first set of test the liquid air freshener system comprises a liquid air freshener composition according to the present invention. The different emulsions merely differ in the perfume oils. The evaporation test was conducted with 90 g samples each emulsion and 8×24 cm plastic fiber reed sticks. The test was carried out over a period of 50 to 60 days.

For comparison, the test was repeated using a liquid air freshener composition based on dipropylene glycol methyl ether (Dowanol DPM).

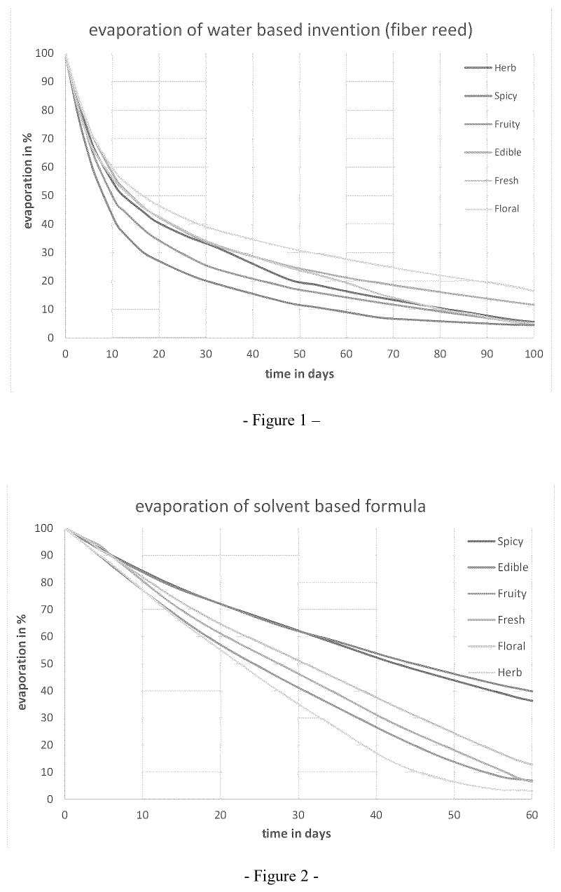

FIG. 1 and FIG. 2 illustrate the obtained experimental results.

FIG. 1 Evaporation test using a liquid air freshener composition according to the present invention.

FIG. 2 Evaporation test using a liquid air freshener composition based on dipropylene glycol methyl ether.

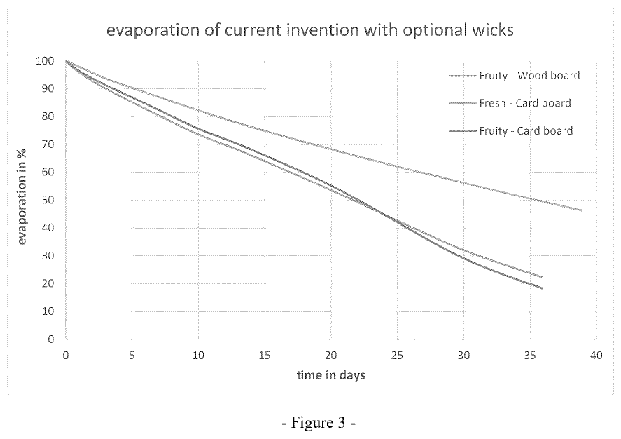

FIG. 3 Evaporation test using diffusers made from different material (wood board and card board).

A completely evaporation of the liquid air freshener composition was confirmed. Further, the evaporation the liquid air freshener system according to the invention was a bit slower than the evaporation using a liquid air freshener composition based on dipropylene glycol methyl ether (Dowanol DPM) and similar conditions as same dosage, sample size and reed stick length.

It is important to point out, that that when approx. 10% air freshener composition remains, the air fresheners are visually empty, as the remaining 10% can “hide” in the wick.

The evaporation curves of the emulsion according to the present invention are flatter than those of the DPM variant; nevertheless, the air fresheners according to the invention are indeed empty later than those containing a composition based on DPM.

1. A liquid air freshener system, comprising:
(i) a container;
(ii) a liquid air freshener composition; and
(iii) a diffuser for diffusing the air freshener composition into surrounding air,
wherein the liquid air freshener composition is an oil-in-water emulsion comprising:
(a) an aqueous phase;
(b) an oily phase comprising at least one perfume or aroma substance or perfume oil;
(c) at least one surfactant;
(d) at least one stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches and modified celluloses and mixtures thereof; and optionally
(e) at least one other additive and/or adjuvant; and
the amount of the surfactant is from 0.4 to 5% by weight, based on the total weight of the oily phase, the oil-in-water emulsion having an average oil droplet size of about 1 to about 15 μm.

2. Liquid air freshener system according to claim 1, wherein the oil-in-water emulsion has an average oil droplet size ≥1 μm and <15 μm.

3. Liquid air freshener system according to claim 1, wherein the proportion of the at least one perfume or aroma substance or perfume oil of the oil-in-water emulsion is 0.1 to 75% by weight, in particular 3 to 8% by weight, relative to the total weight of the oil-in-water emulsion.

4. Liquid air freshener system according to claim 1, wherein the at least one surfactant of the oil-in-water emulsion has an HLB value of 8 to 18, in particular an HLB value of 10 to 14.

5. Liquid air freshener system according to claim 1, wherein the at least one surfactant of the oil-in-water emulsion is selected from the group consisting of non-ionic surfactants, anionic surfactants, amphoteric surfactants, and mixtures thereof.

6. Liquid air freshener system according to claim 1, wherein the oil-in-water emulsion comprises:
25 to 99.9% by weight, in particular 89 to 96% by weight, of the aqueous phase;
0.1 to 75% by weight, in particular 3 to 8% by weight, of the oily phase comprising at least one perfume or aroma substance or perfume oil;
0.004 to 18% by weight, in particular 0.3 to 1.3% by weight of surfactant;
up to 1.5% by weight, in particular 0.6 to 1.1% by weight of stabiliser; and optionally
up to 1% by weight, in particular 0.1 to 0.9% by weight of additives and/or adjuvants;
based on the total weight of the oil-in-water emulsion.

7. Liquid air freshener system according to claim 1, wherein the diffuser is a natural or synthetic porous material that allows the diffusion of the air freshener composition.

8. Liquid air freshener system according to claim 1, wherein the diffuser comprises one or more wicks, reeds, or natural or synthetic porous diffusers.

9. Liquid air freshener system according to claim 1, wherein the diffuser is selected from the group consisting of a wick, a reed diffuser, wooden or card board.

10. Liquid air freshener system according to claim 9, wherein the reed diffuser is made from a material selected from the group consisting of wick, reed diffuser, wooden or card board.

11. Liquid air freshener system according to claim 1, wherein the air freshener system further comprises removable means for preventing diffusion of the air freshener composition into surrounding air.

12. An oil-in-water emulsion as a liquid air freshener composition in a liquid air freshener system, the oil-in-water emulsion comprising:
(a) an aqueous phase;
(b) an oily phase comprising at least one perfume or aroma substance or perfume oil;
(c) at least one surfactant;
(d) at least one stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches and modified celluloses, and mixtures thereof; and optionally
(e) at least one other additive and/or adjuvant;
wherein the amount of the surfactant is from 0.4 to 5% by weight, based on the total weight of the oily phase, and the oil-in-water emulsion having an average oil droplet size of about 1 to about 15 μm.

13. The emulsion according to claim 12, wherein the liquid air freshener system is a wick device or a reed diffuser.

14. An oil-in-water emulsion comprising:
(a) an aqueous phase;
(b) an oily phase comprising at least one perfume or aroma substance or perfume oil;
(c) at least one surfactant;
(d) at least one stabiliser, selected from the group consisting of polyacrylate or polymethacrylate thickener, xanthan gum, gellan gum, guar gum, alginic acid, alginate, agar-agar, carrageenan, welan gum, locust bean gum, tragacanth, gum arabic, pectins, polyoses, starch, dextrin, gelatine, casein, modified starches and modified celluloses, and mixtures thereof; and optionally
(e) at least one other additive and/or adjuvant;
wherein the amount of the surfactant is from 0.4 to 5% by weight, based on the total weight of the oily phase, the oil-in-water emulsion having an average oil droplet size ≥1 μm and <15 μm.

15. A method for perfuming and/or refreshing ambient air, comprising diffusing the liquid air freshener composition according to claim 1 with the diffuser.
